(19) United States
(12) Patent Application Publication
(10) Pub. No.: US 2011/0028508 A1
(43) Pub. Date: Feb. 3, 2011

(54) NOVEL PROCESS FOR THE PREPARATION OF SCOPINE ESTERS

(75) Inventors: Abhay Gaitonde, Maharashtra (IN); Bindu Manojkumar, Maharashtra (IN); Dattatraya Shinde, Maharashtra (IN); Sinderpal Tank, Maharashtra (IN)

Correspondence Address: FULBRIGHT & JAWORSKI L.L.P.;Attn: MN IP Docket, 600 Congress Avenue, Suite 2400, Austin, TX, 78701, US

(73) Assignees: Gemerocs [UK] Limited, Hertfordshire (GB); Mylan India Private Limited, Maharashtra (IN)

(21) Appl. No.: 12/810,231

(22) PCT Filed: Jan. 9, 2009
(86) PCT No.: PCT/GB2009/050014

§ 371 Date: Oct. 20, 2010

(30) Foreign Application Priority Data
Jan. 10, 2008 (IN) 77/KOL/2008

(51) Int. Cl. A61K 31/437; C07D 491/08; A61P 11/00; A61P 11/06; A61P 11/08

(52) U.S. Cl. 514/291; 546/91

(57) ABSTRACT

The present invention relates to novel processes for the preparation of scopine esters and their quaternary salts. In particular, the present invention relates to a process for the preparation of tiotropium bromide, pharmaceutical compositions comprising tiotropium bromide and the use of such compositions in the treatment of respiratory disorders.

### CROSS-REFERENCE TO RELATED APPLICATION(S)

This application is a Section 371 National Stage Application of International No. PCT/GB2009/050014, filed 9 Jan. 2009 and published as WO 2009/087419 A1 on 16 Jul. 2009, which claims priority from the IN Patent Application No. 77/KOL/2008, filed 10 Jan. 2008, the contents of which are incorporated herein in their entirety for all purposes.

### FIELD OF THE INVENTION

The present invention relates to novel processes for the preparation of scopine esters and their quaternary salts. In particular, the present invention relates to a process for the preparation of tiotropium bromide, pharmaceutical compositions comprising tiotropium bromide and the use of such compositions in the treatment of respiratory disorders.

### BACKGROUND OF THE INVENTION

Tiotropium bromide (1), first disclosed in European Patent Application EP418716, is a highly effective anticholinergic agent with a specificity for muscarinic receptors and it is presently approved for the treatment of respiratory disorders, such as asthma or chronic obstructive pulmonary disease (COPD), including chronic bronchitis and emphysema.

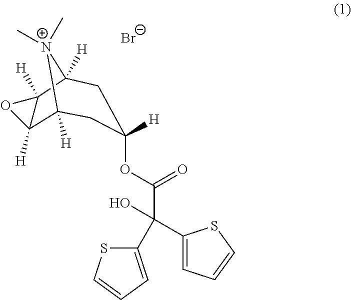

Tiotropium bromide is used in low (microgram) therapeutic doses and it is therefore particularly necessary to develop an industrial process for the commercial preparation of tiotropium bromide which ensures that the product is prepared not only in a good, economical yield but also with exceptional purity.

A process for the preparation of tiotropium bromide was first reported in EP418716. This method of synthesising tiotropium bromide describes, in a first step, the transesterification reaction of scopine (2) with methyl di(2-thienyl)glycolate (4) to form the di(2-thienyl)glycolic acid scopine ester (3), which we will refer to in this application as tiotropium base. The ester (3) is then quaternised with methyl bromide to form tiotropium bromide. However, hazardous reagents such as sodium metal are used for the transesterification step to form tiotropium base (3). In addition, the yields for the preparation of the tiotropium base (3) are low with an HPLC purity around 45-50%- the remaining impurity being di(2-thienyl)glycolic acid (5). The reported process is also inconvenient as the tiotropium base (3) needs to be isolated and purified before quaternisation to afford tiotropium bromide (1).

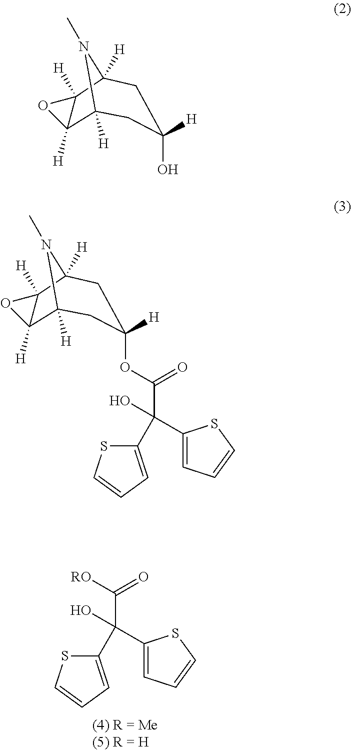

Alternative processes, reported in US patents U.S. Pat. No. 6,486,321, U.S. Pat. No. 6,506,900, U.S. Pat. No. 6,610,849 and U.S. Pat. No. 6,747,153, describe the preparation of tiotropium bromide starting from tropenol hydrochloride. However, these processes are not convenient as they are complex procedures involving a number of synthetic steps and require an epoxidation reaction later in the synthetic route to form the scopine ester moiety.

An alternative process, reported in US patent U.S. Pat. No. 6,747,154, describes the preparation of tiotropium bromide via a direct coupling reaction between di(2-thienyl)glycolic acid and the quaternised derivative scopine methyl bromide. Although this is a short, direct synthesis, the method requires the use of expensive coupling agents such as carbonyldiimidazole, carbonyldi-1,2,4-triazole, ethyldimethylaminopropylcarbodiimide or dicyclohexyl-carbodiimide. In addition, there are other disadvantages to this method as the reaction proceeds at low (sub-zero) temperature, hazardous reagents such as lithium hydride have to be employed, and yields of the purified product are modest.

The processes reported in the prior art, as described above, are not very efficient or convenient for commercial manufacture of pure product and an alternative method is required.

### DEFINITIONS

For the purposes of the present invention, an “alkyl” group is defined as a monovalent saturated hydrocarbon, which may be straight-chained or branched, or be or include cyclic groups. An alkyl group may optionally be substituted, and may optionally include one or more heteroatoms N, O or S in its carbon skeleton. Preferably an alkyl group is straight-chained or branched. Preferably an alkyl group is not substituted. Preferably an alkyl group does not include any heteroatoms in its carbon skeleton. Examples of alkyl groups are methyl, ethyl, n-propyl, propyl, n-butyl, i-butyl, t-butyl, n-pentyl, cyclopentyl, cyclohexyl and cycloheptyl groups. Preferably an alkyl group is a C1-12 alkyl group, preferably a C1-6 alkyl group. Preferably a cyclic alkyl group is a C3-12 cyclic alkyl group, preferably a C5-7 cyclic alkyl group.

An “alkenyl” group is defined as a monovalent hydrocarbon, which comprises at least one carbon-carbon double bond, which may be straight-chained or branched, or be or include cyclic groups. An alkenyl group may optionally be substituted, and may optionally include one or more heteroatoms N, O or S in its carbon skeleton. Preferably an alkenyl group is straight-chained or branched. Preferably an alkenyl group is not substituted. Preferably an alkenyl group does not include any heteroatoms in its carbon skeleton. Examples of alkenyl groups are vinyl, allyl, but-1-enyl, but-2-enyl, cyclohexenyl and cycloheptenyl groups. Preferably an alkenyl group is a C2-12 alkenyl group, preferably a C2-6 alkenyl group. Preferably a cyclic alkenyl group is a C3-12 cyclic alkenyl group, preferably a C5-7 cyclic alkenyl group.

An “alkynyl” group is defined as a monovalent hydrocarbon, which comprises at least one carbon-carbon triple bond, which may be straight-chained or branched, or be or include cyclic groups. An alkynyl group may optionally be substituted, and may optionally include one or more heteroatoms N, O or S in its carbon skeleton. Preferably an alkynyl group is straight-chained or branched. Preferably an alkynyl group is not substituted. Preferably an alkynyl group does not include any heteroatoms in its carbon skeleton. Examples of alkynyl groups are ethynyl, propargyl, but-1-ynyl and but-2-ynyl groups. Preferably an alkynyl group is a C2-12 alkynyl group, preferably a C2-6 alkynyl group. Preferably a cyclic alkynyl group is a C3-12 cyclic alkynyl group, preferably a C5-7 cyclic alkynyl group.

An “aryl” group is defined as a monovalent aromatic hydrocarbon. An aryl group may optionally be substituted, and may optionally include one or more heteroatoms N, O or S in its carbon skeleton. Preferably an aryl group is not substituted. Preferably an aryl group does not include any heteroatoms in its carbon skeleton. Examples of aryl groups are phenyl, naphthyl, anthracenyl, phenanthrenyl, thienyl and furyl groups. Preferably an aryl group is a C4-14 aryl group, preferably a C6-10 aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

An “alkoxy” group is defined as a —O-alkyl, —O-alkenyl, —O-alkynyl, —O-aryl, —O-arylalkyl, —O-arylalkenyl, —O-arylalkynyl, —O-alkylaryl, —O-alkenylaryl or —O-alkynylaryl group. Preferably an “alkoxy” group is a —O-alkyl or —O-aryl group. More preferably an “alkoxy” group is a —O-alkyl group.

A “halo” group is a fluoro, chloro, bromo or iodo group.

For the purposes of this invention, an optionally substituted group may be substituted with one or more of —F, —Cl, —Br, —I, —CF3, —CCl3, —CBr3, —CI3, —OH, —SH, —NH2, —CN, —NO2, —COOH, —Ra—O—Rb, —Ra—S—Rb, —Ra—N(Rb)2, —Ra—N(Rb)3+, —Ra—P(Rb)2, —Ra—Si(Rb)3, —Ra—CO—Rb, —Ra—CO—ORb, —RaO—CO—Rb, —Ra—CO—N(Rb)2, —Ra—NRb—CO—Rb, —RaO—CO—ORb, —RaO—CO—N(Rb)2, —Ra—NRb—CO—ORb, —Ra—NRb—CO—N(Rb)2, —Ra—CS—Rb or —Rb. In this context, —Ra— is independently a chemical bond, a C1-C10 alkylene, C2-C10 alkenylene or C2-C10 alkynylene group. —Rb is independently hydrogen, unsubstituted C1-C6 alkyl or unsubstituted C6-C10 aryl. Preferably an optionally substituted group may be substituted with one or more of C1-C4 alkyl, C1-C4 alkoxy, hydroxy, halo or haloalkyl, all unsubstituted. Optional substituent(s) are not taken into account when calculating the total number of carbon atoms in the parent group substituted with the optional substituent(s). Preferably a substituted group comprises 1, 2 or 3 substituents, more preferably 1 or 2 substituents, and even more preferably 1 substituent.

For the purposes of the present invention, a compound is “substantially pure”, if it comprises less than 1% impurity by HPLC, preferably less than 0.5%, preferably less than 0.3%, preferably less than 0.2%, preferably less than 0.1%.

### SUMMARY OF THE INVENTION

The present invention provides a process for preparing highly pure tiotropium and related compounds by the most convenient and shortest route, which avoids the use of hazardous and/or environmentally unsuitable reagents.

The present invention also provides an efficient, simple and non-hazardous process for the preparation of tiotropium bromide (1), tiotropium base (3) and related compounds.

A first aspect of the present invention provides a process for the preparation of the scopine ester I or its quaternary salt II:

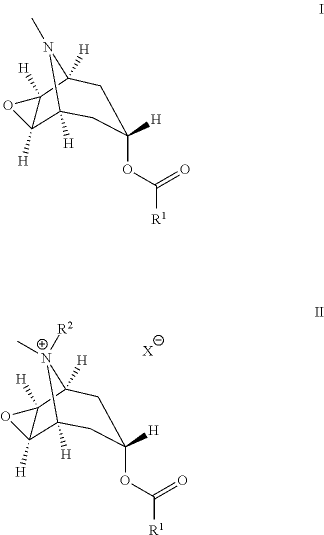

comprising transesterification of scopine, or a salt thereof, with a suitable carboxylic ester R1CO2R3; wherein R1 and R2 independently represent hydrogen, alkyl, alkenyl, alkynyl, optionally substituted aryl, or optionally substituted arylalkyl, arylalkenyl, arylalkynyl, alkylaryl, alkenylaryl or alkynylaryl; R3 represents alkyl, alkenyl, alkynyl, optionally substituted aryl, or optionally substituted arylalkyl, arylalkenyl, arylalkynyl, alkylaryl, alkenylaryl or alkynylaryl; and X represents a pharmaceutically acceptable anion.

In a preferred process R1 is represented by formula III, wherein R4, R5 and R6 independently represent hydrogen, hydroxy, halo, alkoxy, alkyl, hydroxyalkyl, alkenyl, alkynyl, optionally substituted aryl, or optionally substituted arylalkyl, arylalkenyl, arylalkynyl, alkylaryl, alkenylaryl or alkynylaryl.

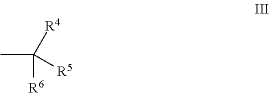

Preferably R4 and/or R5 represent aryl, wherein the aryl group can be selected from phenyl, naphthyl, thienyl and furyl, which may optionally be mono- or disubstituted by one or two groups selected from C1-C4 alkyl, C1-C4 alkoxy, hydroxy, halo or haloalkyl. Most preferably the aryl group is 2-thienyl.

Preferably R6 represents hydroxy, C1-C4 alkyl, C1-C4 alkoxy, hydroxyalkyl, halo or haloalkyl.

Most preferably, R4 is 2-thienyl, R5 is 2-thienyl and R6 is hydroxy.

Preferably R2 represents hydrogen or C1-C4 alkyl, more preferably methyl.

Preferably R3 represents C1-C4 alkyl, and most preferably R3 represents methyl.

Preferably, X represents halo, methanesulfonate, toluenesulfonate or trifluoromethanesulfonate. Most preferably X represents a bromo.

Most preferably when forming the quaternary salt II, R2 is methyl and X is bromo.

Preferably the scopine is used in the form of a salt, preferably an acid addition salt, and most preferably in the form of its hydrochloride salt.

Preferably, the transesterification reaction is performed in the presence of a base, preferably an organic base, preferably an organic amine base. The organic amine base is preferably a trialkylamine such as triethylamine or diisopropylethylamine, or a heterocyclic amine such as 1,8-diazabicyclo[5.4.0]undec-7-ene (DBU), 1,5-diazabicyclo[4.3.0]non-5-ene (DBN), 1,4-diazabicyclo[2.2.2]octane (Dabco), pyridine or 4-(dimethylamino)pyridine (DMAP). Most preferably, the organic amine base is DBU. Preferably 1-5 equivalents of the organic amine base are used relative to the scopine or the salt thereof, preferably 1-3 equivalents of the organic amine base are used.

Additionally a further base may be used for the transesterification reaction. Preferably the further base is an inorganic base, preferably a hydride such as NaH, KH or CaH2. Preferably the further base is NaH. Preferably 1-2 equivalents of the further base are used relative to the scopine or the salt thereof.

If two bases are used, they may be used in any order, i.e. the further base may be added to the reaction mixture before, after and/or simultaneous with the first base. Without wishing to be bound by theory, the base(s) are believed to deprotonate the scopine hydroxyl group or a protonated reaction intermediate. Moreover, if the scopine is used in the form of a salt, the base(s), in particular the inorganic base, are believed to liberate scopine free base in situ.

The reaction temperature used in the transesterification step is preferably in the range of 30 to 90° C., more preferably in the range of 40 to 70° C., and more preferably in the range of 50 to 70° C. Most preferably the reaction is carried out at about 60° C.

Preferably, the process according to the first aspect of the invention is carried out such that formation of the quaternary salt II is obtained without purification and/or isolation of ester I.

Preferably, the transesterification reaction is carried out in a polar aprotic solvent, such as a solvent selected from dimethylformamide, dimethylsulfoxide, acetonitrile or N-methylpyrrolidine. Preferably, the solvent is dimethylformamide.

Preferably, the scopine ester I or its quaternary salt II are obtained in an HPLC purity of greater than 95%, preferably greater than 98%, preferably greater than 99%, preferably greater than 99.5%, preferably greater than 99.7%, preferably greater than 99.8%, more preferably greater than 99.9%.

Preferably, the scopine ester I or its quaternary salt II are obtained in a yield of greater than 50%, preferably greater than 55%, more preferably greater than 60%.

A second aspect of the invention provides substantially pure tiotropium base.

A third aspect of the invention provides substantially pure tiotropium bromide. Preferably the tiotropium bromide is suitable for use in medicine, preferably for treating or preventing a respiratory disorder, such as asthma or COPD, wherein the COPD can include chronic bronchitis and emphysema.

A fourth aspect of the invention provides tiotropium base or tiotropium bromide prepared by a process according to the first aspect of the invention. Preferably the tiotropium base or tiotropium bromide is substantially pure. Preferably the tiotropium bromide is suitable for use in medicine, preferably for treating or preventing a respiratory disorder, such as asthma or COPD, wherein the COPD can include chronic bronchitis and emphysema.

A fifth aspect of the invention provides a pharmaceutical composition comprising tiotropium bromide prepared according to the first aspect of the invention. Preferably, the pharmaceutical composition is suitable for use in a dry powder inhaler (DPI), an aqueous nebulizer, or a pressurized metered dosage inhaler (pMDI).

A sixth aspect of the invention provides for the use of tiotropium bromide according to the third or fourth aspect of the invention, or the use of the composition according to the fifth aspect of the invention, for the manufacture of a medicament for the treatment or prevention of respiratory disorders, such as asthma or COPD, wherein the COPD can include chronic bronchitis and emphysema.

A seventh aspect of the invention provides a method of treating or preventing a respiratory disorder, comprising administering to a patient in need thereof a therapeutically or prophylactically effective amount of tiotropium bromide according to the third or fourth aspect of the invention, or a therapeutically or prophylactically effective amount of the composition according to the fifth aspect of the invention. Preferably the respiratory disorder is asthma or COPD, wherein the COPD can include chronic bronchitis and emphysema. Preferably the patient is a mammal, preferably a human.

### DETAILED DESCRIPTION OF THE INVENTION

Surprisingly, it has been found that tiotropium base (3) and tiotropium bromide (1) can be obtained in substantially pure form when synthesised by the efficient and more advantageous process of the present invention. A preferred embodiment of the present invention is outlined in Scheme 1.

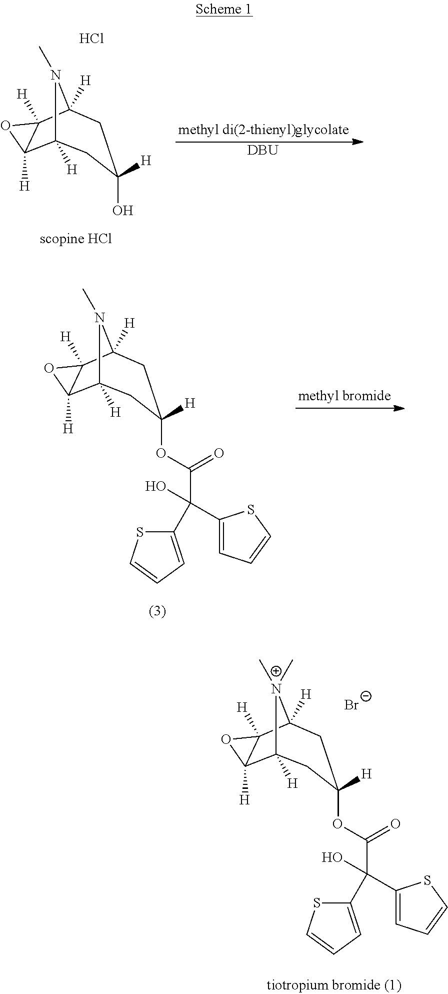

The process outlined in Scheme 1 is a novel process for the preparation of tiotropium base (3) and is very advantageous as the use of DBU (1,8-diazabicyclo[5.4.0]undec-7-ene) as base gives a dramatic improvement in the purity from 45-50% (obtained when using prior art processes) to >99.6% by HPLC. The overall yield is also improved over the prior art. The solvent used is preferably DMF and the reaction is preferably carried out at about 60° C.

Preferably, the solvent used in the transesterification step is DMF, but alternatively the solvent used can be dimethylsulfoxide, acetonitrile or N-methylpyrrolidine.

The reaction temperature used in the transesterification step is preferably in the range of 30 to 90° C., more preferably in the range of 40 to 70° C., and more preferably in the range of 50 to 70° C. Most preferably the reaction is carried out at about 60° C.

The tiotropium base (3) formed by the present invention is so pure that it can surprisingly be quaternised, for example with methyl bromide, without isolation and purification to afford a highly pure quaternary salt product. This is a huge benefit in a commercial operation as it saves very significantly on time and cost if a purification and/or isolation step can be avoided.

The organic bases used in the present invention are preferably organic amines, most preferably trialkylamines such as diisopropylethylamine or triethylamine, or a heterocyclic amine such as 1,8-diazabicyclo[5.4.0]undec-7-ene (DBU), 1,5-diazabicyclo[4.3.0]non-5-ene (DBN), 1,4-diazabicyclo[2.2.2]octane (Dabco), pyridine or 4-(dimethylamino)pyridine (DMAP). The organic base used is most preferably DBU.

Preferably the scopine is used in the form of its hydrochloride salt, but alternatively it can be used in the form of the free base or any other suitable salt, such as other mineral acid addition salts (e.g. HBr or HI) or organic acid addition salts (e.g. acetate, benzoate, propionate, maleate, fumarate, oxalate, besylate, mesylate, tosylate, citrate or salicylate).

Other preferred embodiments of the first aspect of the invention are the preparation of scopolamine [represented by ester I, wherein R1 is represented by formula III and R4 is hydrogen, R5 is phenyl and R6 is hydroxymethyl (CH2OH)]; scopolamine hydrogen bromide [represented by salt II, wherein R1 is represented by formula III and R4 is hydrogen, R5 is phenyl, R6 is hydroxymethyl, R2 is hydrogen and X is bromo]; oxitropium bromide [represented by salt II, wherein R1 is represented by formula III and R4 is hydrogen, R5 is phenyl, R6 is hydroxymethyl, R2 is ethyl and X is bromo]; and cimetropium bromide [represented by salt II, wherein R1 is represented by formula III and R4 is hydrogen, R5 is phenyl, R6 is hydroxymethyl, R2 is —CH2-cyclopropyl and X is bromo].

A fifth aspect of the invention provides a pharmaceutical composition comprising tiotropium bromide prepared according to the first aspect of the invention. Preferably, the pharmaceutical composition is suitable for use in a dry powder inhaler (DPI), an aqueous nebulizer, or a pressurized metered dosage inhaler (pMDI).

The DPI compositions of the present invention preferably contain, in addition to the active substance, one or more of the following physiologically acceptable excipients: monosaccharides (e.g. glucose or arabinose), disaccharides (e.g. lactose, sucrose, maltose), oligo- and polysaccharides (e.g. dextrane), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly in the form of their hydrates. For the purposes of the present invention, lactose is a particularly preferred excipient, while lactose monohydrate is most particularly preferred.

Preferably the pMDI uses HFA 134a, HFA 227 or mixtures thereof as propellant gas.

The pharmaceutical composition according to the fifth aspect of the present invention can also be a solution, suspension or a solid oral dosage form if so desired. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant and optionally further comprises at least one excipient selected from colouring agents, adsorbents, surfactants, film formers and plasticizers.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

The pharmaceutical compositions of the present invention preferably contain about 0.001 to 20% tiotropium bromide in admixture with a physiologically acceptable excipient. Preferred compositions contain 0.01 to 10% of tiotropium bromide, more preferred are compositions which contain 0.01 to 2% of tiotropium bromide, and most preferred are compositions which contain 0.04 to 0.8% of tiotropium bromide.

The following paragraphs enumerated consecutively from 1 through 49 provide for various aspects of the present invention. In one embodiment, the present invention provides:

1. A process for the preparation of scopine ester I or its quaternary salt II:

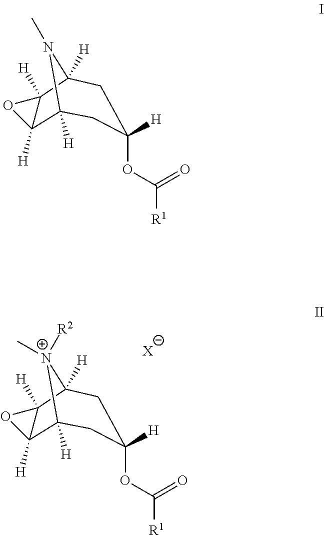

comprising transesterification of scopine, or a salt thereof, with a suitable carboxylic ester represented by formula R1CO2R3; wherein R1 and R2 independently represent hydrogen, alkyl, alkenyl, alkynyl, optionally substituted aryl, or optionally substituted arylalkyl, arylalkenyl, arylalkynyl, alkylaryl, alkenylaryl or alkynylaryl; R3 represents alkyl, alkenyl, alkynyl, optionally substituted aryl, or optionally substituted arylalkyl, arylalkenyl, arylalkynyl, alkylaryl, alkenylaryl or alkynylaryl; and X represents a pharmaceutically acceptable anion.

2. A process according to paragraph 1, wherein R1 is represented by formula III:

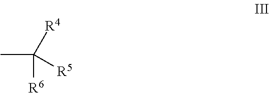

wherein R4, R5 and R6 independently represent hydrogen, hydroxy, halo, alkoxy, alkyl, hydroxyalkyl, alkenyl, alkynyl, optionally substituted aryl, or optionally substituted arylalkyl, arylalkenyl, arylalkynyl, alkylaryl, alkenylaryl or alkynylaryl.

3. A process according to paragraph 2, wherein R4 and/or R5 represent aryl.

4. A process according to paragraph 3, wherein the aryl group is selected from phenyl, naphthyl, thienyl and furyl, which may optionally be mono- or disubstituted by one or two groups selected from C1-C4 alkyl, C1-C4 alkoxy, hydroxy, halo or haloalkyl.

5. A process according to paragraph 4, wherein the aryl group is 2-thienyl.

6. A process according to any one of paragraphs 2 to 5, wherein R6 represents hydroxy, C1-C4 alkyl, C1-C4 alkoxy, hydroxyalkyl, halo or haloalkyl.

7. A process according to any one of paragraphs 2 to 6, wherein R4 is 2-thienyl, R5 is 2-thienyl and R6 is hydroxyl.

8. A process according to any one of the preceding paragraphs, wherein R2 represents hydrogen or C1-C4 alkyl.

9. A process according to any one of the preceding paragraphs, wherein R3 represents C1-C4 alkyl.

10. A process according to paragraph 9, wherein R3 represents methyl.

11. A process according to any one of the preceding paragraphs, wherein X represents a halo, a methanesulfonate, a toluenesulfonate or a trifluoromethanesulfonate group.

12. A process according to paragraph 11, wherein X represents a bromo group.

13. A process according to any one of the preceding paragraphs, wherein R2 is methyl and X is bromo.

14. A process according to any one of the preceding paragraphs, wherein the scopine is used in the form of its hydrochloride salt.

15. A process according to any one of the preceding paragraphs, wherein the transesterification reaction is performed in the presence of a base.

16. A process according to paragraph 15, wherein the base is an organic base.

17. A process according to paragraph 16, wherein the organic base is an organic amine base.

18. A process according to paragraph 17, wherein the organic amine base is a trialkylamine or a heterocyclic amine.

19. A process according to paragraph 18, wherein the organic amine base is selected from triethylamine, diisopropylethylamine, 1,8-diazabicyclo[5.4.0]undec-7-ene (DBU), 1,5-diazabicyclo[4.3.0]non-5-ene (DBN), 1,4-diazabicyclo[2.2.2]octane (Dabco), pyridine or 4-(dimethylamino)pyridine (DMAP).

20. A process according to paragraph 19, wherein the organic amine base is DBU.

21. A process according to any one of paragraphs 15 to 20, wherein a further base is used.

22. A process according to paragraph 21, wherein the further base is an inorganic base.

23. A process according to paragraph 22, wherein the inorganic base is a hydride.

24. A process according to paragraph 23, wherein the hydride is NaH, KH or CaH2.

25. A process according to paragraph 24, wherein the hydride is NaH.

26. A process according to any one of paragraphs 21 to 25, wherein the scopine is used in the form of a salt.

27. A process according to paragraph 26, wherein the further base is used to liberate scopine free base in situ.

28. A process according to any one of the preceding paragraphs, wherein formation of the quaternary salt II is carried out without purification and/or isolation of ester I.

29. A process according to any one of the preceding paragraphs, wherein the transesterification reaction is carried out in dimethylformamide.

30. A process according to any one of the preceding paragraphs, wherein the scopine ester I or its quaternary salt II are obtained in an HPLC purity of greater than 95%.

31. A process according to any one of the preceding paragraphs, wherein the scopine ester I or its quaternary salt II are obtained in a yield of greater than 50%.

32. Substantially pure tiotropium base (3).

33. Substantially pure tiotropium bromide (1).

34. Tiotropium base (3) or tiotropium bromide (1) prepared by a process according to any one of paragraphs 1 to 31.

35. Tiotropium base (3) or tiotropium bromide (1) according to paragraph 34, which is substantially pure.

36. Tiotropium bromide (1) according to any one of paragraphs 33 to 35, for use in medicine.

37. Tiotropium bromide (1) according to paragraph 36, for treating or preventing a respiratory disorder.

38. Tiotropium bromide (1) according to paragraph 37, wherein the respiratory disorder comprises asthma or COPD.

39. Tiotropium bromide (1) according to paragraph 38, wherein the COPD is chronic bronchitis or emphysema.

40. A pharmaceutical composition comprising tiotropium bromide (1) according to any one of paragraphs 33 to 39.

41. The pharmaceutical composition according to paragraph 40, which is suitable for use in a dry powder inhaler (DPI), an aqueous nebulizer, or a pressurized metered dosage inhaler (pMDI).

42. Use of tiotropium bromide (1) according to any one of paragraphs 33 to 39, or use of the composition according to paragraph 40 or 41, for the manufacture of a medicament for the treatment or prevention of a respiratory disorder.

43. The use according to paragraph 42, wherein the respiratory disorder comprises asthma or COPD.

44. The use according to paragraph 43, wherein the COPD is chronic bronchitis or emphysema.

45. A method of treating or preventing a respiratory disorder, comprising administering to a patient in need thereof a therapeutically or prophylactically effective amount of tiotropium bromide (1) according to any one of paragraphs 33 to 39, or a therapeutically or prophylactically effective amount of the composition according to paragraph 40 or 41.

46. The method according to paragraph 45, wherein the respiratory disorder comprises asthma or COPD.

47. The method according to paragraph 46, wherein the COPD is chronic bronchitis or emphysema.

48. The method according to any one of paragraphs 45 to 47, wherein the patient is a mammal.

49. The method according to paragraph 48, wherein the patient is a human.

The following examples are provided to illustrate the present invention and should not be construed as limiting thereof.

### EXAMPLES

### Tiotropium Base (3)

Scopine HCl was taken in DMF (5 vol), cooled to 5° C., and NaH (1.7 eq) was added slowly maintaining the temperature at 5° C. The reaction was stirred for 1 hour at 10° C. and DBU (1 eq) and methyl di(2-thienyl)glycolate (1 eq) were added. The reaction was heated to 60° C. for 1 hour and a second portion of DBU (2 eq) was added. The reaction was heated for a further 4 hours at 60° C. and monitored by TLC. After completion of the reaction, the mixture was cooled to 5° C. and a solution of conc. HCl (2.5 vol) in cold water (10 vol) at 10° C. (pH 2) was added. The mixture was washed with toluene and basified with aqueous sodium carbonate (7.5 eq) to pH 10 and extracted with DCM (3×10 vol). The combined DCM layer was washed with water (3×10 vol) and DCM was distilled under vacuum (150 mbar) at 30° C. The product was obtained as light brown solid. Molar Yield=60%; HPLC purity=98%.

The crude base (3) was recrystallized from acetonitrile (5 vol). Yield of crystallization=86%; HPLC purity>99.8%.

Tiotropium bromide (1)

The purified tiotropium base (3) was dissolved in DCM (10 vol) and acetonitrile (3 vol) and purged with methyl bromide gas at a pressure of 10 kg/cm2 for 20 minutes. The solution was kept at 25-30° C. for 30 hours. The precipitated solid was filtered and washed with DCM (20 vol). Drying of the solid at 25-30° C. under vacuum gave the product as a white solid. Molar Yield=97.76%; HPLC purity=99.83%.

1H-NMR (300 MHz, CD3OD): 7.45 (2H dd), 7.23 (2H dd), 7.00 (2H dd), 5.27 (1H t), 4.60 (1H br s OH), 3.35 (8H m), 3.10 (2H s), 2.85 (2H dt), 2.10 (2H d).

MS: 392.3 (M+1)

Alternative Process for the Preparation of Tiotropium Bromide (1) without Isolation of Tiotropium Base (3)

Scopine HCl was taken in DMF (5 vol), cooled to 5° C., and NaH (1.7 eq) was added slowly maintaining the temperature at 5° C. The reaction mixture was stirred for 1 hour at 10° C. and DBU (1 eq) and methyl di(2-thienyl)glycolate (1 eq) were added. The reaction was heated to 60° C. for 1 hour and a second lot of DBU (2 eq) was added. The reaction mixture was heated for a further 4 hours at 60° C. and monitored by TLC. After completion of the reaction, the mixture was cooled to 5° C. and a solution of conc. HCl (2.5 vol) in cold water (10 vol) at 10° C. (pH 2) was added. The mixture was washed with toluene (1 vol) and the aqueous layer was basified with saturated sodium carbonate (7.5 eq) solution to pH 10 and extracted with DCM (3×10 vol). The combined DCM layer was washed with water (3×10 vol) and dried over anhydrous sodium sulfate.

To the DCM solution, acetonitrile was added and the mixture was purged with methyl bromide gas at a pressure of 3 kg/cm2 for 30 minutes. The solution was kept at 25-30° C. for 30 hours. The precipitated solid was filtered and washed with DCM (20 vol). Drying of the solid at 25-30° C. under vacuum gave the product as a white solid. Molar yield=41%; HPLC purity=98.66%.

1. A process for the preparation of scopine ester I or its quaternary salt II: 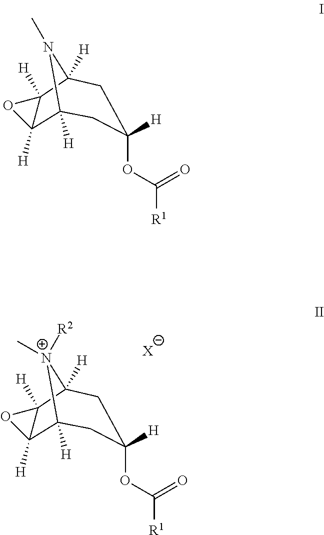
comprising transesterification of scopine, or a salt thereof, with a suitable carboxylic ester represented by formula R¹CO₂R⁵; wherein R¹ and R² independently represent hydrogen, alkyl, alkenyl, alkynyl, optionally substituted aryl, or optionally substituted arylalkyl, arylalkenyl, arylalkynyl, alkylaryl, alkenylaryl or alkynylaryl; R³ represents alkyl, alkenyl, alkynyl, optionally substituted aryl, or optionally substituted arylalkyl, arylalkenyl, arylalkynyl, alkylaryl, alkenylaryl or alkynylaryl; and X represents a pharmaceutically acceptable anion.

2. A process according to claim 1, wherein R¹ is represented by formula III: 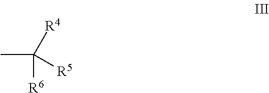
wherein R⁴, R⁵ and R⁶ independently represent hydrogen, hydroxy, halo, alkoxy, alkyl, hydroxyalkyl, alkenyl, alkynyl, optionally substituted aryl, or optionally substituted arylalkyl, arylalkenyl, arylalkynyl, alkylaryl, alkenylaryl or alkynylaryl.

3. A process according to claim 2, wherein R⁴ and/or R⁵ represent aryl.

4. A process according to claim 3, wherein the aryl group is selected from phenyl, naphthyl, thienyl and furyl, which may optionally be mono- or disubstituted by one or two groups selected from C₁-C₄ alkyl, C₁-C₄ alkoxy, hydroxy, halo or haloalkyl.

5. A process according to claim 4, wherein the aryl group is 2-thienyl.

6. A process according to any one of claims 2 to 5, wherein R⁶ represents hydroxy, C₁-C₄ alkyl, C₁-C₄ alkoxy, hydroxyalkyl, halo or haloalkyl.

7. A process according to any one of claims 2 to 6, wherein R⁴ is 2-thienyl, R⁵ is 2-thienyl and R⁶ is hydroxyl.

8. A process according to any one of the preceding claims, wherein R² represents hydrogen or C₁-C₄ alkyl.

9. A process according to any one of the preceding claims, wherein R³ represents C₁-C₄ alkyl.

10. A process according to claim 9, wherein R³ represents methyl.

11. A process according to any one of the preceding claims, wherein X represents a halo, a methanesulfonate, a toluenesulfonate or a trifluoromethanesulfonate group.

12. A process according to claim 11, wherein X represents a bromo group.

13. A process according to any one of the preceding claims, wherein R² is methyl and X is bromo.

14. A process according to any one of the preceding claims, wherein the scopine is used in the form of its hydrochloride salt.

15. A process according to any one of the preceding claims, wherein the transesterification reaction is performed in the presence of a base.

16. A process according to claim 15, wherein the base is an organic base.

17. A process according to claim 16, wherein the organic base is an organic amine base.

18. A process according to claim 17, wherein the organic amine base is a trialkylamine or a heterocyclic amine.

19. A process according to claim 18, wherein the organic amine base is selected from triethylamine, diisopropylethylamine, 1,8-diazabicyclo[5.4.0]undec-7-ene (DBU), 1,5-diazabicyclo[4.3.0]non-5-ene (DBN), 1,4-diazabicyclo[2.2.2]octane (Dabco), pyridine or 4-(dimethylamino)pyridine (DMAP).

20. A process according to claim 19, wherein the organic amine base is DBU.

21. A process according to any one of claims 15 to 20, wherein a further base is used.

22. A process according to claim 21, wherein the further base is an inorganic base.

23. A process according to claim 22, wherein the inorganic base is a hydride.

24. A process according to claim 23, wherein the hydride is NaH, KH or CaH₂.

25. A process according to claim 24, wherein the hydride is NaH.

26. A process according to any one of claims 21 to 25, wherein the scopine is used in the form of a salt.

27. A process according to claim 26, wherein the further base is used to liberate scopine free base in situ.

28. A process according to any one of the preceding claims, wherein formation of the quaternary salt II is carried out without purification and/or isolation of ester I.

29. A process according to any one of the preceding claims, wherein the transesterification reaction is carried out in dimethylformamide.

30. A process according to any one of the preceding claims, wherein the scopine ester I or its quaternary salt II are obtained in an HPLC purity of greater than 95%.

31. A process according to any one of the preceding claims, wherein the scopine ester I or its quaternary salt II are obtained in a yield of greater than 50%.

32. Substantially pure tiotropium base (3).

33. Substantially pure tiotropium bromide (1).

34. Tiotropium base (3) or tiotropium bromide (1) prepared by a process according to any one of claims 1 to 31.

35. Tiotropium base (3) or tiotropium bromide (1) according to claim 34, which is substantially pure.

36. Tiotropium bromide (1) according to any one of claims 33 to 35, for use in medicine.

37. Tiotropium bromide (1) according to claim 36, for treating or preventing a respiratory disorder.

38. Tiotropium bromide (1) according to claim 37, wherein the respiratory disorder comprises asthma or COPD.

39. Tiotropium bromide (1) according to claim 38, wherein the COPD is chronic bronchitis or emphysema.

40. A pharmaceutical composition comprising tiotropium bromide (1) according to any one of claims 33 to 39.

41. The pharmaceutical composition according to claim 40, which is suitable for use in a dry powder inhaler (DPI), an aqueous nebulizer, or a pressurized metered dosage inhaler (pMDI).

42. Use of tiotropium bromide (1) according to any one of claims 33 to 39, or use of the composition according to claim 40 or 41, for the manufacture of a medicament for the treatment or prevention of a respiratory disorder.

43. The use according to claim 42, wherein the respiratory disorder comprises asthma or COPD.

44. The use according to claim 43, wherein the COPD is chronic bronchitis or emphysema.

45. A method of treating or preventing a respiratory disorder, comprising administering to a patient in need thereof a therapeutically or prophylactically effective amount of tiotropium bromide (1) according to any one of claims 33 to 39, or a therapeutically or prophylactically effective amount of the composition according to claim 40 or 41.

46. The method according to claim 45, wherein the respiratory disorder comprises asthma or COPD.

47. The method according to claim 46, wherein the COPD is chronic bronchitis or emphysema.

48. The method according to any one of claims 45 to 47, wherein the patient is a mammal.

49. The method according to claim 48, wherein the patient is a human.
